(19) United States
(12) Patent Application Publication
(10) Pub. No.: US 2025/0032760 A1
(43) Pub. Date: Jan. 30, 2025

(54) ONE-HANDED INSERTION DEVICE FOR GUIDEWIRE AND IMPROVED SYRINGE WITH GUIDEWIRE

(71) Applicant: EVA INNOVATIONS, INC., Aventura, FL (US)

(72) Inventors: Daniel B. Stone, Aventura, FL (US); Jeffrey A. Panozzo, Naples, FL (US)

(73) Assignee: EVA INNOVATIONS, INC., Aventura, FL (US)

(21) Appl. No.: 18/667,465

(22) Filed: May 17, 2024

Related U.S. Application Data
(60) Provisional application No. 63/515,273, filed on Jul. 24, 2023.

(51) Int. Cl. A61M 25/09

(52) U.S. Cl. CPC A61M 25/09041

(57) ABSTRACT

A device for one-handed insertion of a guidewire includes a mounting/gripping member and an actuator carriage. A syringe mounted on the mounting/gripping member has a barrel and a plunger containing a valve assembly. A guidewire is installed through a passage in the plunger. The valve assembly engages the guidewire with at least one elastic seal in an interference fit. The carriage has a guidewire feed and a trigger. The trigger can slide the carriage axially along the mounting/gripping member, which moves the plunger forwardly and backwardly. While a user holds the mounting/gripping member, the trigger can be moved with a digit and another digit of the same hand engages the guidewire between the digit and the guidewire feed and advances it through the syringe, sensing the degree of resistance encountered by a portion of the guidewire.

### CROSS-REFERENCE TO RELATED APPLICATION

This application claims the benefit of priority of U.S. provisional application No. 63/515,273, filed Jul. 24, 2023, the contents of which are herein incorporated by reference.

### TECHNICAL FIELD

This application is directed to a device for inserting a guidewire into the lumen/cavity of a patient; in particular, a device adapted for one-handed operation and an improved syringe with a guidewire installed having a valve assembly in the syringe plunger which prevents substantial leakage during operation.

### BACKGROUND

WO 2018/089758 discloses a device for one-handed insertion of a guidewire, comprising a body having: a grip associated with said body, a mount formed in said body for a syringe of the type having: a plunger, a plunger flange and a guidewire passage through said plunger and a hollow needle co-linear with said guidewire passage through said plunger, wherein said mount is adapted to receive and retain a barrel of a syringe; and an actuator carriage slidably coupled to said body via a guideway, wherein said guideway is formed in either said body or said actuator carriage, the actuator carriage having attached thereto: a trigger, a flange trap adapted to grasp the plunger flange of a syringe, a guidewire feed surface between said rear guidewire control tube wherein said feed surface and said trigger are disposed relative to said grip such that when the grip is engaged by the digits of one hand, a digit of said one hand is able to urge the trigger of said actuator carriage rearwardly and/or forwardly, and another digit of said one hand is able to engage the guidewire between the digit of said one hand and the guidewire feed surface, such that when the grip is engaged by the digits of one hand, a digit of said one hand is able to advance the guidewire through said syringe by engaging the guidewire between the digit of said one hand and the guidewire feed surface, sensing the degree of resistance encountered by a portion of said guidewire, such as the tip, with another digit of said one hand being able to urge the trigger of said actuator carriage rearwardly and/or forwardly, drawing said plunger rearwardly and/or forwardly while digits of said one hand continue to engage said grip.

Other references of interest include U.S. Pat. No. 4,813,938 to Raulerson; United States (syringe); United States Patent Publication No. 2015/0314104 of Almansouri et al. (guidewheel); U.S. Pat. No. 6,485,428 to Enk Col 2, lines 45-57; U.S. Pat. No. 6,217,558 to Zandini et al., Col. 12, lines 54-67.

With respect to valve configurations generally, see U.S. Pat. No. 8,262,623 to Nijland et al., as well as United States Patent Publication No. 2017/0002941 of Nijland et al.

Existing devices for guidewire insertion generally rely on a syringe of the class disclosed in U.S. Pat. No. 4,813,938 to Raulerson; however such devices require that a needle be placed in the lumen of a blood vessel before the guidewire is installed in the syringe due to air/fluid leakage during operation. This complicates operation of the device, requiring additional manipulation of the components. There thus exists a need in the art for a device adapted for one-handed operation that can be operated with the guidewire pre-installed in the syringe. It is also noted that existing devices are unnecessarily large and unwieldly, oftentimes exhibiting a “lever” effect which is difficult to control.

### SUMMARY OF INVENTION

The present invention provides a compact design for a guidewire insertion device adapted for one-handed operation including a syringe having a plunger, a plunger flange and a guidewire passage through said plunger as well as a valve assembly in the guidewire passage. The valve assembly is configured to prevent substantial air/fluid leakage during needle placement, even with the guidewire pre-installed through the plunger. The valve assembly has at least one elastic seal around the installed guidewire, where the guidewire exhibits a degree of interference with the elastic seal in the range of about 15% to 80%. A substantial degree of interference between the seal and the guidewire was found not to interfere with movement of the guidewire through the device.

Further improvements include a more compact design, the improved device is shorter in length than prior devices; typically about 6.5″ as compared to 8″ and 9″ for prior art devices. The trigger on the carriage has moved forward-toward the needle. This allows for improved handling of the device as the operator's hand is closer to the needle than prior devices. This change allows for significant improvement of performing the procedure as compared to the Raulerson syringe as the operator has increased control during the procedure. The improved design has a significant decrease in the ‘lever’ effect as compared to the Raulerson syringe and prior art devices.

The new design allows only a minimal amount of air into the barrel of the syringe when in use, even though it still uses the same interior perforate metal cylinder in the plunger as in a conventional Raulerson syringe. That is, the new syringe is built to ensure minimal air in the barrel of the syringe upon pulling back on the plunger. Currently, when using the Raulerson syringe, the guidewire is not placed inside the syringe until the needle is set in the lumen of a vessel. When implementing the new device, the guidewire is situated inside the syringe from the start of the procedure. If you place the guidewire in a conventional Raulerson syringe and pull back on the plunger, most of the contents in the syringe is a backflow of air, that is, when the guidewire is situated past/through the valve assembly in the Raulerson syringe, upon pulling back on the plunger, a significant amount of air (more than half of the barrel of the syringe) will enter the Raulerson syringe rather than blood/liquid. A large amount of air in the syringe barrel, rather than blood/liquid, is not acceptable as the blood/liquid represents the positioning of the needle within the lumen. Conversely, when a user pulls back on the new design plunger, only a minimal amount of air enters the syringe barrel.

The new design of the syringe also prevents liquid from exiting via the back of the plunger handle while a guidewire is pre-loaded in the syringe through the use of a dome valve assembly with a back-up seal. The dome valve assembly is located inside the syringe and creates a seal around the guidewire by applying tension around it. The tension applied does not hinder the operator's ability to advance the guidewire into the lumen/cavity but is sufficient to prevent liquid/air passage. The seal is made of any suitable elastic material, such as, without limitation, a rubber, a thermoplastic elastomer, a medical grade silicone, or any combination thereof. Most preferably the seal is made of an ISO 10993 medical Silicone. Other designs of syringes for this application are not capable of allowing for a pre-loaded guidewire because the valves used do not apply the proper tension around the guidewire.

Further details are provided in the annexed drawings and the text which follows.

### BRIEF DESCRIPTION OF DRAWINGS

The invention is described in detail below with reference to the drawings, wherein like numerals designate similar parts and wherein:

FIG. 1 is a view in perspective of the inventive insertion device with the guidewire installed, without a needle;

FIG. 2 is a view in perspective of the device of FIG. 1 with the guidewire extended;

FIG. 3 is a view in perspective of the device of FIG. 1, without the syringe installed;

FIG. 4 is a view in perspective and longitudinal section of the device of FIG. 1;

FIG. 5A is a side view of the device of FIG. 1, without a syringe, wherein the carriage is in a forwardmost position;

FIG. 5B is a side view of the device of FIG. 1, without a syringe, wherein the carriage is in a fully retracted position;

FIG. 6 is a sectional view of the syringe of FIG. 2 with the guidewire installed through the syringe;

FIG. 7 is a sectional view the valve assembly components in the plunger of FIG. 1 in a partially assembled and uncompressed state; and

FIG. 8 is a view in elevation of the plunger in the device of FIG. 1, wherein the valve assembly is installed in a compressed state.

### DETAILED DESCRIPTION

The present invention is described in detail below in connection with the annexed drawings. Terminology has its ordinary meaning; mm means millimeters, cc or ml means cubic centimeters and so forth.

“Degree of interference” and like technology refers to the difference in guidewire diameter to seal diameter divided by seal diameter times 100%, or

((guidewire diameter−seal diameter)=seal diameter)×100%

“Air leak value” is expressed in percent as (air leak volume/syringe volume)×100%. Air leak volume is determined by drawing a full syringe volume from a reservoir of deionized water at 25° C. while disposing the syringe at approximately 45° from vertical with the guidewire installed in the syringe throughout the draw process. Thus, a 3 cc syringe which exhibits 3/5 cc of air leakage with the guidewire installed has an air leak value of (0.6/3)×100% or 20%.

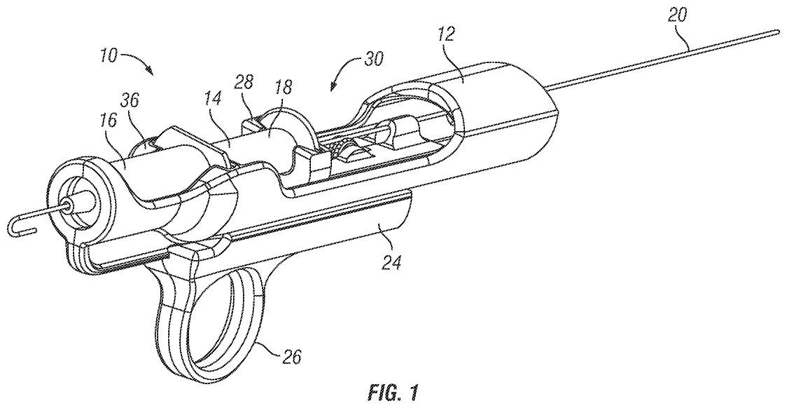

Referring to FIG. 1, there is illustrated a device 10 for the one-handed insertion of a guidewire 20 including a mounting/gripping member 12 having disposed thereon a syringe 14 with a barrel 16 and a plunger 18. Plunger 18 has further features, discussed below.

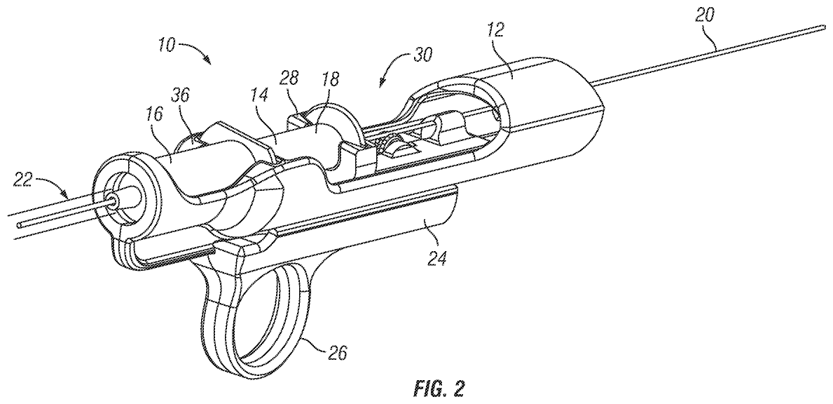

FIG. 1 shows device 10 with the guidewire 20 installed with “J” bend in the guidewire extending through the device, while FIG. 2 shows device 10 with guidewire 20 extending through device 10 and a hollow needle indicated schematically at 22.

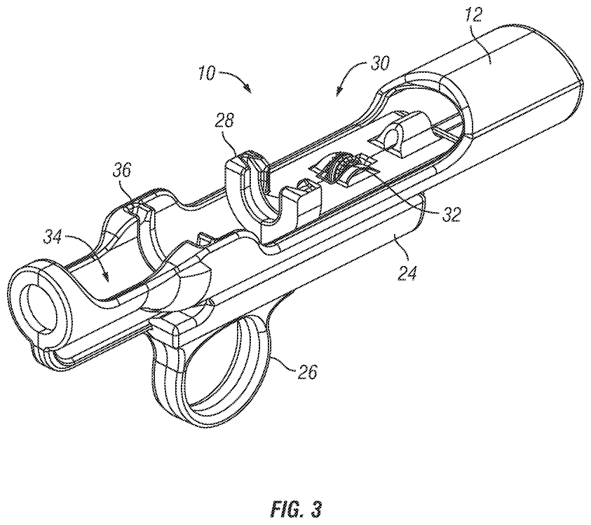

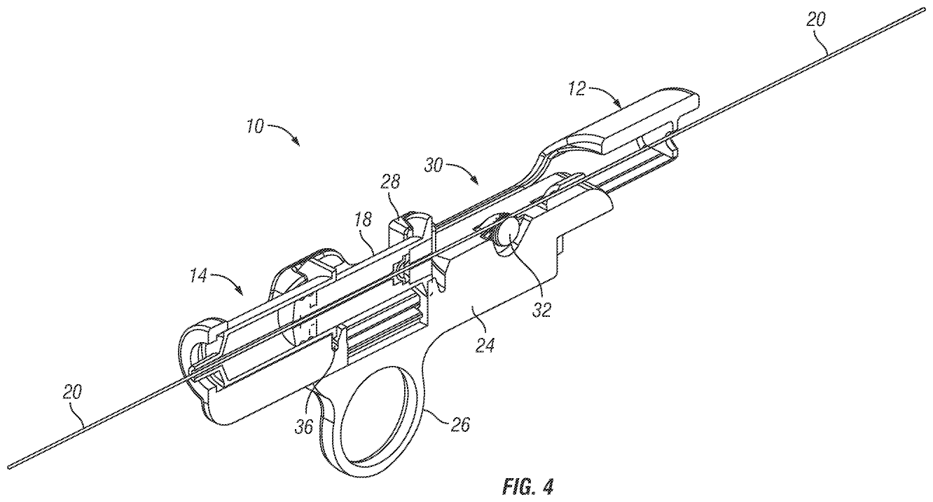

The various features of device 10 are further illustrated in FIGS. 3, 4 and 5.

Device 10 includes a carriage 24 slidably engaged to mounting/gripping member 12 having a trigger 26 and a flange trap 28 for a flange in the plunger, as well as a guidewire feed 30, including a guidewheel feed 32 (see FIG. 3).

Mounting/gripping member 12 includes a mounting area 34 for receiving the syringe including a barrel flange trap 36 for receiving the barrel in place on mounting/gripping member 12. Alternatively, barrel 16 may be integral with mounting/gripping member 12.

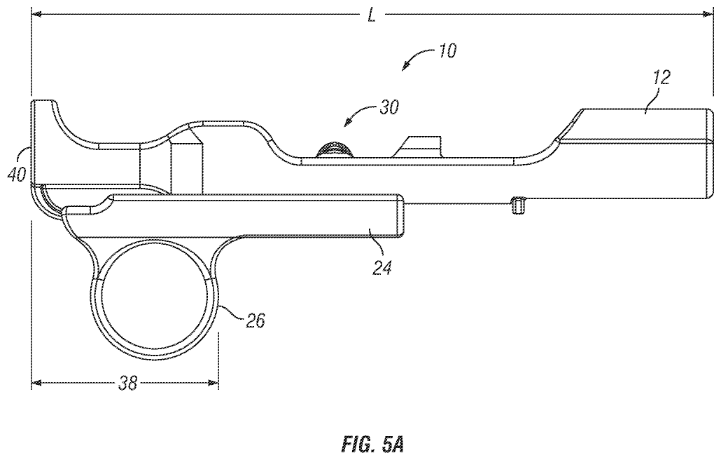

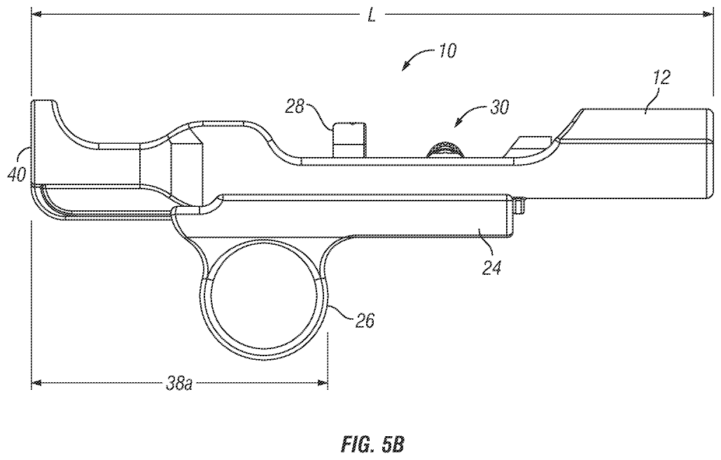

Gripping member 12 has a length, L, of 7″ or less, typically from about 3″ to about 7″. FIG. 5A shows carriage 24 in a forwardmost position where a distance 38 of trigger 26 is at most about 2.25″; suitably, from about 0″ to about 4.0″. Distance 38 is measured from an end 40 of the mounting/gripping member 12 to the innermost projection of trigger 26 in its forwardmost position as indicated on FIG. 5A. Note that the carriage is considerably shorter than the mounting/gripping member.

In FIG. 5B carriage 24 is in a fully retracted position, wherein distance 38a is measured from end 40 of the mounting/gripping member 12 to a rearwardmost projection of trigger 26 in its fully retracted position. Distance 38a is anywhere from about 0.25″ to about 5″.

As alluded to earlier, a raison d'être of the present invention is to allow clinicians to practice a Seldinger technique using only one hand for both needle placement as well as for manipulation of the guidewire while using the other hand to position a visualization device, such as an ultrasound, thus enabling the clinician to ensure that the ultrasound is placed properly for optimum viewing of the needle/guidewire as it enters the lumen while freeing assistants to perform other functions as needed. In many cases, where a central line is needed, time will absolutely be of the essence and the ability of the clinician to insert the line with minimal delay will be of the utmost importance.

In the Seldinger technique, a syringe having a beveled needle is inserted into the body with the bevel oriented in such a fashion that the point of the bevel first enters the skin so that when the guidewire exits the bore of the syringe, it will be oriented generally parallel to the longitudinal axis of the body passageway in question or in some known other orientation if a guidewire is to be introduced into some other bodily structure. Where body conformation allows, it is usually preferable that the needle of the syringe make about a 45° angle (or typically less) with the surface of the skin with the point of the bevel engaging the skin first. The fixtures of the present invention enable the clinician to apply negative pressure by pulling back slightly on the plunger of the syringe with one hand whereas in the conventional technique, the use of 2 hands can be somewhat awkward or clumsy. In many cases, the clinician will be able to manipulate the plunger using a single digit, perhaps the index finger, on the hand holding the fixture.

Once the guidewire is passing through the bore of the needle, the clinician may have increased ability to perceive the amount of resistance (tactile perception) being encountered by the guidewire while also being able to more expeditiously use the ultrasound continuously to ensure the guidewire enters into, and remains in, the lumen of the vessel or other desired location without using the other hand or requiring an assistant. The device of the present subject matter enables the clinician to perceive subtle variations in the degree of resistance, indicative of the tissue/vessel through which the guidewire is passing. This can be extremely significant as it avoids the contingencies in which the clinician might be tempted to withdraw the guidewire slightly to re-orient it in the lumen of the vessel. In particular, if such a withdrawal is not handled properly, there is a possibility that the guidewire may be cut by the bevel of the needle particularly when the guidewire is curved at the tip or is for some other reason bent. In many cases, it is of course conventional for the guidewire to have a “J” shaped tip to guard against inadvertent perforation of a vessel wall.

After the guidewire is properly positioned, with the fixture of the present invention, the clinician moves the fixture rearwardly and only is required to begin using his other hand to restrain the guidewire as the bevel of the needle exits the body since the grip of surrounding tissue will be sufficient to hold the guidewire in place while the fixture is being withdrawn.

Once the guidewire has been properly positioned, and the means of inserting it have been cleared away, the clinician using the fixture of the present invention will proceed generally in accordance with the conventional procedure.

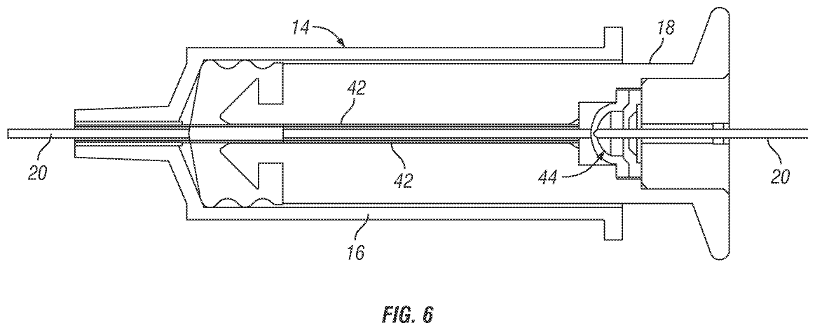

The present invention further facilitates practicing a one-handed Seldinger technique in that the device simplifies operation considerably due to its compact design and because the guidewire is pre-installed in the device, which requires improved sealing around the guidewire to prevent air and other fluid leakage as discussed below in connection with FIGS. 6, 7 and 8.

Syringe 14 has a perforate conduit 42 for receiving guidewire 20 secured within barrel 16 which slidably accommodates plunger 18.

A valve assembly 44 is disposed in the plunger. Assembly 44 includes a forward dome valve 46 having a slit seal 48, similar to a duck-bill valve, and a back-up valve 50 with a cylindrical seal 52. Both valves are most preferably made of ISO 10993 medical silicone.

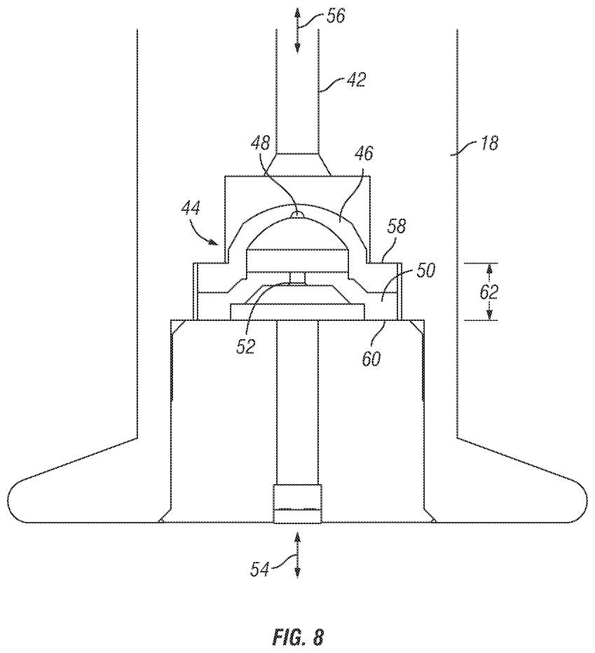

Guidewire 20 has a degree of interference with seal 52 of anywhere from about 15% to 80%, with from about 35% to about 55% being more preferred. Guidewire 20 is installed in the syringe as indicated by arrows 54, 56 on FIG. 8. A guidewire with a diameter of 0.80 mm through a valve with a cylindrical seal having a diameter of 0.55 mm has a degree of interference of about 45% with respect to the seal.

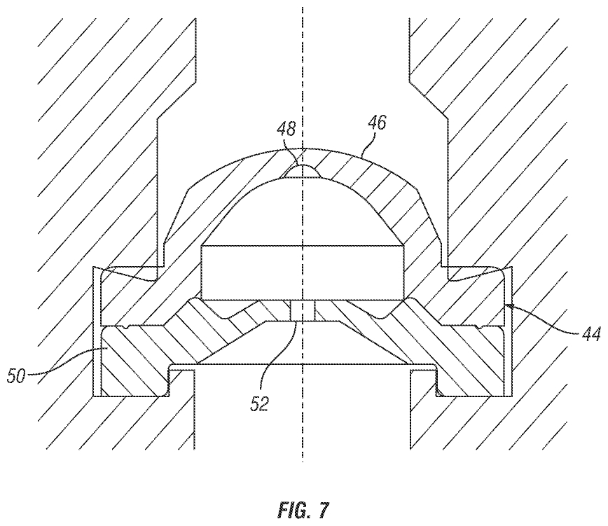

Besides the degree of interference of the guidewire with the elastic seal, it is also important not to distort the seals during assembly of the plunger. In FIG. 7, the valves are shown in an un-compressed state, whereas in FIG. 8 the valves are shown in an assembled, compressed state within the plunger, wherein the valves are compressed together in a cavity within the plunger. It is particularly important, indeed critical, that the valves in their compressed state are not distorted to lose their seal shapes. To this end, retaining surfaces 58, 60 do not compress the valves more than about 7.5-15% from their original dimensions; regardless of assembly method, whether threaded and screwed together or snap-fit with appropriate features on the plunger components. To this end, surfaces 58, 60 are of a predetermined axial distance 62 with respect to one another of no less than about 85%-92.5% of the thickness of the peripheries of the valve assembly components prior to compression.

The invention further encompasses the following embodiments:

Embodiment 1. A device for one-handed insertion of a guidewire comprising:
(a) a mounting/gripping member having disposed thereon a syringe with a barrel and a plunger with a guidewire passage therethrough and a valve assembly located within the plunger;
(b) an actuator carriage slidably engaged to the mounting/gripping member having a guidewire feed and a trigger, the carriage also being engaged to the plunger of the syringe so as to axially move the plunger of the syringe forwardly and backwardly by manipulation of the trigger and sliding motion of the carriage with respect to the mounting/gripping member; and
(c) a guidewire installed through the plunger,
wherein the guidewire engages the valve assembly which has at least one elastic seal in an interference fit with the guidewire and wherein said guidewire feed and said trigger are disposed relative to said mounting/gripping member such that when the mounting/gripping member is engaged by the digits of one hand, a digit of said one hand is able to urge the trigger of said actuator carriage rearwardly and/or forwardly, and another digit of said one hand is able to engage the guidewire between the digit of said one hand and the guidewire feed, such that when the mounting/gripping member is engaged by the digits of one hand, a digit of said one hand is able to advance the guidewire through said syringe by engaging the guidewire between the digit of said one hand and the guidewire feed, sensing the degree of resistance encountered by the tip of said guidewire, with another digit of said one hand being able to urge the trigger of said actuator carriage rearwardly and/or forwardly, drawing said plunger rearwardly and/or forwardly while digits of said one hand continue to engage said mounting/gripping member.

Embodiment 2. The device according to Embodiment 1, wherein the at least one elastic seal in an interference fit with the guidewire has a degree of interference with the guidewire from about 15% to about 80%.

Embodiment 3. The device according to Embodiment 1 or 2, wherein the at least one elastic seal in an interference fit with the guidewire has a degree of interference with the guidewire of at least about 25%.

Embodiment 4. The device according to any of the foregoing embodiments, wherein the at least one elastic seal in an interference fit with the guidewire has a degree of interference with the guidewire of at least about 35%.

Embodiment 5. The device according to any of the foregoing embodiments, wherein the at least one elastic seal in an interference fit with the guidewire has a degree of interference with the guidewire of at least about 45%.

Embodiment 6. The device according to any of the foregoing embodiments, wherein the at least one elastic seal in an interference fit with the guidewire has a degree of interference with the guidewire of at least about 55%.

Embodiment 7. The device according to any of the foregoing embodiments, wherein the at least one elastic seal in an interference fit with the guidewire has a degree of interference with the guidewire of at least about 65%.

Embodiment 8. The device according to any of the foregoing embodiments, wherein the at least one elastic seal in an interference fit with the guidewire is made of a rubber.

Embodiment 9. The device according to any of the foregoing embodiments, wherein the at least one elastic seal in an interference fit with the guidewire is made of a thermoplastic elastomer.

Embodiment 10. The device according to any of Embodiments 1-8, wherein the at least one elastic seal in an interference fit with the guidewire is made of an ISO 10993 medical Silicone.

Embodiment 11. The device according to any of the foregoing embodiments, wherein the syringe exhibits an air leak value of 30% or less.

Embodiment 12. The device according to any of the foregoing embodiments, wherein the syringe exhibits an air leak value of 25% or less.

Embodiment 13. The device according to any of the foregoing embodiments, wherein the syringe exhibits an air leak value of 20% or less.

Embodiment 14. The device according to any of the foregoing embodiments, wherein the syringe exhibits an air leak value of from less than 1% to about 30%.

Embodiment 15. The device according to any of the foregoing embodiments, wherein the mounting/gripping member has a length of about 7 inches or less.

Embodiment 16. The device according to any of the foregoing embodiments, wherein the mounting/gripping member has a length of from about 3 inches to about 7 inches.

Embodiment 17. The device according to any of the foregoing embodiments, wherein the trigger of the actuator carriage is no more than about 4 inches, preferably no more than about 2.25 inches rearwardly disposed from a front end of the mounting/gripping member when the actuator carriage is disposed in a forewardmost position with respect to the mounting/gripping member.

Embodiment 18. The device according to any of the foregoing embodiments, wherein the trigger of the actuator carriage is from about 0 to no more than about 4 inches, preferably no more than about 2.25 inches rearwardly disposed from a front end of the mounting/gripping member when the actuator carriage is disposed in a forewardmost position with respect to the mounting/gripping member.

Embodiment 19. A device for one-handed insertion of a guidewire comprising:
(a) a mounting/gripping member having disposed thereon a syringe with a barrel and a plunger with a guidewire passage therethrough and a valve assembly located within the plunger;
(b) an actuator carriage slidably engaged to the mounting/gripping member having a guidewire feed and a trigger, the carriage also being engaged to the plunger of the syringe so as to axially move the plunger of the syringe forwardly and backwardly by manipulation of the trigger and sliding motion of the carriage with respect to the mounting/gripping member; and
(c) a guidewire adapted to be installed through the plunger,
wherein, when installed in the plunger, the guidewire engages the valve assembly which has at least one elastic seal in an interference fit with the guidewire and wherein said guidewire feed and said trigger are disposed relative to said mounting/gripping member such that when the mounting/gripping member is engaged by the digits of one hand, a digit of said one hand is able to urge the trigger of said actuator carriage rearwardly and/or forwardly, and another digit of said one hand is able to engage the guidewire between the digit of said one hand and the guidewire feed, such that when the mounting/gripping member is engaged by the digits of one hand, a digit of said one hand is able to advance the guidewire through said syringe by engaging the guidewire between the digit of said one hand and the guidewire feed, sensing the degree of resistance encountered by the tip of said guidewire, with another digit of said one hand being able to urge the trigger of said actuator carriage rearwardly and/or forwardly, drawing said plunger rearwardly and/or forwardly while digits of said one hand continue to engage said mounting/gripping member.

Embodiment 20. A syringe with a guidewire installed comprising:
(a) a syringe with a barrel and a plunger with a guidewire passage therethrough and a valve assembly located within the plunger; and
(b) a guidewire installed through the plunger, wherein the guidewire engages the valve assembly which has at least one elastic seal in an interference fit with the guidewire.

Embodiment 21. The syringe and guidewire according to Embodiment 20, wherein the at least one elastic seal in an interference fit with the guidewire has a degree of interference with the guidewire from about 15% to about 80%.

Embodiment 22. The syringe and guidewire according to Embodiment 20 or 21, wherein the at least one elastic seal in an interference fit with the guidewire has a degree of interference with the guidewire of at least about 25%.

Embodiment 23. The syringe and guidewire according to any of Embodiments 20 to 22, wherein the at least one elastic seal in an interference fit with the guidewire has a degree of interference with the guidewire of at least about 35%.

Embodiment 24. The syringe and guidewire according to any of Embodiments 20 to 23, wherein the at least one elastic seal in an interference fit with the guidewire has a degree of interference with the guidewire of at least about 45%.

Embodiment 25. The syringe and guidewire according to any of Embodiments 20 to 24, wherein the at least one elastic seal in an interference fit with the guidewire has a degree of interference with the guidewire of at least about 55%.

Embodiment 26. The syringe and guidewire according to any of Embodiments 20 to 25, wherein the at least one elastic seal in an interference fit with the guidewire has a degree of interference with the guidewire of at least about 65%.

Embodiment 27. The syringe and guidewire according to any of Embodiments 20 to 26, wherein the at least one elastic seal in an interference fit with the guidewire is made of a rubber.

Embodiment 28. The syringe and guidewire according to any of Embodiments 20 to 27, wherein the at least one elastic seal in an interference fit with the guidewire is made of a thermoplastic elastomer.

Embodiment 29. The syringe and guidewire according to any of Embodiments 20 to 27, wherein the at least one elastic seal in an interference fit with the guidewire is made of an ISO 10993 medical Silicone.

Embodiment 30. The syringe and guidewire according to any of Embodiments 20 to 29, wherein the syringe exhibits an air leak value of 30% or less.

Embodiment 31. The syringe and guidewire according to any of Embodiments 20 to 30, wherein the syringe exhibits an air leak value of 25% or less.

Embodiment 32. The syringe and guidewire according to any of Embodiments 20 to 31, wherein the syringe exhibits an air leak value of 20% or less.

Embodiment 33. The syringe and guidewire according to any of Embodiments 20 to 32, wherein the syringe exhibits an air leak value of from less than 1% to about 30%.

Embodiment 34. The syringe and guidewire according to any of Embodiments 20 to 33, wherein the valve assembly includes a forward dome valve with a slit seal and a rearward back-up valve with a cylindrical seal.

Embodiment 35. The syringe and guidewire according to Embodiment 34, wherein the dome valve and back-up valve are made of a rubber.

Embodiment 36. The syringe and guidewire according to Embodiment 35, wherein the dome valve and back-up valve are made of a thermoplastic elastomer.

Embodiment 37. The syringe and guidewire according to Embodiment 35, wherein the dome valve and back-up valve are made of an ISO 10993 medical Silicone.

Embodiment 38. A syringe and a guidewire comprising:
(a) a syringe with a barrel and a plunger with a guidewire passage therethrough and a valve assembly located within the plunger; and
(b) a guidewire adapted to be installed through the plunger,
wherein, when the guidewire is installed in the plunger, the guidewire engages the valve assembly which has at least one elastic seal in an interference fit with the guidewire.

While the invention has been described in detail, modifications within the spirit and scope of the invention will be readily apparent to those of skill in the art. Such modifications are also to be considered as part of the present invention. In view of the foregoing discussion, relevant knowledge in the art and references discussed above in connection with the Background of the Invention and Detailed Description, the disclosures of which are all incorporated herein by reference, further description is deemed unnecessary. In addition, it should be understood that aspects of the invention and portions of various embodiments may be combined or interchanged either in whole or in part. Furthermore, those of ordinary skill in the art will appreciate that the foregoing description is by way of illustration only and is not intended to limit the invention.

1. A device for one-handed insertion of a guidewire comprising:
(a) a mounting/gripping member having disposed thereon a syringe with a barrel and a plunger with a guidewire passage therethrough and a valve assembly located within the plunger;
(b) an actuator carriage slidably engaged to the mounting/gripping member having a guidewire feed and a trigger, the actuator carriage also being engaged to the plunger of the syringe so as to axially move the plunger of the syringe forwardly and backwardly by manipulation of the trigger and sliding motion of the actuator carriage with respect to the mounting/gripping member; and
(c) a guidewire installed through the plunger,
wherein the guidewire engages the valve assembly which has at least one elastic seal in an interference fit with the guidewire and wherein said guidewire feed and said trigger are disposed relative to said mounting/gripping member such that when the mounting/gripping member is engaged by one hand, a digit of said one hand is able to urge the trigger of said actuator carriage rearwardly and/or forwardly, and between the digit of said one hand and the guidewire feed, another digit of said one hand is able to engage the guidewire to advance the guidewire through said syringe, sensing the degree of resistance encountered by a portion of said guidewire.

2. The device according to claim 1, wherein the at least one elastic seal in an interference fit with the guidewire has a degree of interference with the guidewire from about 15% to about 80%.

3. The device according to claim 1, wherein the at least one elastic seal in an interference fit with the guidewire has a degree of interference with the guidewire of at least about 25%.

4. The device according to claim 1, wherein the at least one elastic seal in an interference fit with the guidewire is made of a material selected from the group consisting of a rubber, a thermoplastic elastomer, an ISO 10993 medical grade silicone, and any combination thereof.

5. The device according to claim 1, wherein the syringe exhibits an air leak value of 30% or less.

6. The device according to claim 1, wherein the syringe exhibits an air leak value of from less than 1% to about 30%.

7. The device according to claim 1, wherein the mounting/gripping member has a length of about 7 inches or less.

8. The device according to claim 1, wherein the mounting/gripping member has a length of from about 3 inches to about 7 inches.

9. The device according to claim 1, wherein the trigger of the actuator carriage is no more than about 4 inches rearwardly disposed from a front end of the mounting/gripping member when the actuator carriage is disposed in a forewardmost position with respect to the mounting/gripping member.

10. A device for one-handed insertion of a guidewire comprising:
(a) a mounting/gripping member having disposed thereon a syringe with a barrel and a plunger with a guidewire passage therethrough and a valve assembly located within the plunger;
(b) an actuator carriage slidably engaged to the mounting/gripping member having a guidewire feed and a trigger, the actuator carriage also being engaged to the plunger of the syringe so as to axially move the plunger of the syringe forwardly and backwardly by manipulation of the trigger and sliding motion of the actuator carriage with respect to the mounting/gripping member; and
(c) a guidewire adapted to be installed through the plunger,
wherein, when installed in the plunger, the guidewire engages the valve assembly which has at least one elastic seal in an interference fit with the guidewire and wherein said guidewire feed and said trigger are disposed relative to said mounting/gripping member such that when the mounting/gripping member is engaged by one hand, a digit of said one hand is able to urge the trigger of said actuator carriage rearwardly and/or forwardly, and between the digit of said one hand and the guidewire feed, another digit of said one hand is able to engage the guidewire to advance the guidewire through said syringe, sensing the degree of resistance encountered by a portion of said guidewire.

11. A syringe with a guidewire, comprising:
(a) a syringe with a barrel and a plunger with a guidewire passage therethrough and a valve assembly located within the plunger; and
(b) a guidewire installed through the plunger,
wherein the guidewire engages the valve assembly which has at least one elastic seal in an interference fit with the guidewire.

12. The syringe and guidewire according to claim 11, wherein the at least one elastic seal in an interference fit with the guidewire has a degree of interference with the guidewire from about 15% to about 80%.

13. The syringe and guidewire according to claim 11, wherein the at least one elastic seal in an interference fit with the guidewire has a degree of interference with the guidewire of at least about 25%.

14. The syringe and guidewire according to claim 11, wherein the at least one elastic seal in an interference fit with the guidewire is made of a material selected from the group consisting of a rubber, a thermoplastic elastomer, an ISO 10993 medical grade silicone, and any combination thereof.

15. The syringe and guidewire according to claim 11, wherein the syringe exhibits an air leak value of 30% or less.

16. The syringe and guidewire according to claim 11, wherein the syringe exhibits an air leak value of from less than 1% to about 30%.

17. The syringe and guidewire according to claim 11, wherein the valve assembly includes a forward dome valve with a slit seal and a rearward back-up valve with a cylindrical seal.

18. The syringe and guidewire according to claim 17, wherein the forward dome valve and the rearward back-up valve are made of a material selected from the group consisting of a rubber, a thermoplastic elastomer, an ISO 10993 medical grade silicone, and any combination thereof.
